(19) United States
(12) Patent Application Publication
(10) Pub. No.: US 2025/0057723 A1
(43) Pub. Date: Feb. 20, 2025

(54) ELECTRIC MASSAGE COMB WITH ESSENTIAL OIL ATOMIZATION AND INFRARED LIGHT APPARATUS

(71) Applicant: Shenzhen Lunmake Technology Co., Ltd., Shenzhen (CN)

(72) Inventor: Jiwu LONG, Shaoyang (CN)

(73) Assignee: Shenzhen Lunmake Technology Co., Ltd., Shenzhen (CN)

(21) Appl. No.: 18/641,398

(22) Filed: Apr. 21, 2024

(30) Foreign Application Priority Data
Aug. 14, 2023 (CN) 2023221739253

(51) Int. Cl. A61H 11/00; A45D 24/00; A45D 24/22; A45D 34/04; A45D 34/06; A61H 23/02; A61M 11/00

(52) U.S. Cl. CPC A61H 11/00; A45D 24/007; A45D 24/22; A45D 34/04; A45D 34/06; A61H 23/0236; A61M 11/005; A45D 2200/205; A45D 2200/207; A61H 2201/0207; A61H 2201/5007; A61H 2205/021; A61M 2205/052

(57) ABSTRACT

The present utility model provides an electric massage comb with an essential oil atomization and infrared light apparatus, including a main massage comb body, a control circuit board disposed inside the main massage comb body, a cartridge body assembly, a teeth plate at an outer side of the main massage comb body, and a sonic motor for driving the teeth plate to vibrate, where a spray outlet for spraying mist is provided on the teeth plate part, and the cartridge body assembly, the sonic motor, and the control circuit board are electrically connected.

### CROSS-REFERENCE TO RELATED APPLICATIONS

The application claims priority to Chinese patent application No. 2023221739253, filed on Aug. 14, 2023, the entire contents of which are incorporated herein by reference.

### TECHNICAL FIELD

The present utility model relates to the technical field of massage comb products, and in particular, to an electric massage comb with an essential oil atomization and infrared light apparatus that offers good user experience and high utility.

### BACKGROUND

Conventional head massagers or massage combs provide massage by using a brush-based rotary motor with mechanical rolling forces and mechanical squeezing forces. Such a design and operating manner cannot easily reach deep muscles in actual application.

Moreover, conventional massage combs with essential oil deliver the essential oil by using balls according to a hydrodynamic principle, and there is a problem that the essential oil is delivered only from a few places and cannot cover entire scalp and hair.

In addition, the conventional massage combs generally simply adopt mechanical massage, or have only several spots of infrared light irradiation. There is few light and low power, not powerful enough for improving scalp microcirculation.

For example, the utility model patent application No. CN202221641022.2 entitled “Hairdressing Comb With Essential Oil” has specifically disclosed a hairdressing comb with essential oil that includes a main body housing. A vibration assembly, a mist assembly, and main body circuitry are disposed inside the main body housing. A switch is disposed on the main body housing, and the main body circuitry is electrically connected to the switch, the mist assembly, the vibration assembly separately. The bottom of the vibration assembly is connected to a teeth plate fixation frame, the bottom of the teeth plate fixation frame extends under the main body housing, and the bottom of the teeth plate fixation frame is movably connected to a teeth plate. The mist assembly is connected to an air outlet duct for mist to come out, and the air outlet duct runs through, from top to bottom, the main body housing, the teeth plate fixation frame, and the top of the teeth plate, to deliver mist to the teeth plate and then to hair. This hairdressing comb integrates essential oil in a comb body, and the atomized essential oil is blown to the top of head by a fan for essential oil-based hairdressing, offering convenience in use.

However, the technical solution in the conventional technology has the problem described above. To sum up, massage for deep muscles and full care for scalp and hair is an urgent technical issue to be resolved at present in the field of head massage combs. This requires improvement and enhancement of structures and principles for resolving the problem of the conventional technology.

### SUMMARY

A problem in a conventional technology to be resolved in this application is that:

Conventional head massagers or massage combs use a brush-based rotary motor, which cannot easily reach deep muscles in actual application. These massage combs with essential oil deliver the essential oil by using balls according to a hydrodynamic principle, and there is a problem that the essential oil is delivered only from a few places and cannot cover entire scalp and hair. The conventional massage combs generally simply adopt mechanical massage, or have only several spots of infrared light irradiation. There is few light and low power, not powerful enough for improving scalp microcirculation.

The present utility model resolves the technical problems with the following solutions.

An electric massage comb with an essential oil atomization and infrared light apparatus is provided, and includes a main massage comb body, a control circuit board disposed inside the main massage comb body, a cartridge body assembly, a teeth plate at an outer side of the main massage comb body, and a sonic motor for driving the teeth plate to vibrate, where a spray outlet for spraying mist is provided on the teeth plate part, and the cartridge body assembly, the sonic motor, and the control circuit board are electrically connected.

The electric massage comb with the essential oil atomization and infrared light apparatus further includes an air pump that is disposed inside the main massage comb body and that is interconnected to the cartridge body assembly through an air duct, where mist produced by the cartridge body assembly is transferred, through a straight-through hose with pumping of the air pump, to the spray outlet part for spraying.

The teeth plate is connected to an output shaft of the sonic motor and is driven by the sonic motor to vibrate.

Preferably, an infrared light board, or infrared light beads, is further disposed in the main massage comb body, to deepen the mist into scalp with infrared light radiation.

Preferably, the cartridge body assembly includes a cartridge body, a ceramic heating strip inside the cartridge body, and a ceramic heating strip support for fixedly disposing the ceramic heating strip, an air inlet and an air outlet are provided on the cartridge body for a connection to the air pump and mist pumping.

Preferably, a cartridge support for mounting the cartridge body assembly is further disposed in the cartridge body assembly.

One end of the cartridge body is disposed with a lower cartridge casing, and the cartridge body assembly further includes a conductive pin on the lower cartridge casing part and magnet sheets for ensuring a conductive connection effect.

Preferably, a vertical groove corresponding to the spray outlet is provided on the teeth plate, and electric massage comb with an essential oil atomization and infrared light apparatus further includes a spray joint disposed at the vertical groove part, where the spray joint is interconnected to the cartridge body assembly through the straight-through hose.

Preferably, the main massage comb body includes a top casing and a bottom casing, and the teeth plate is disposed near the bottom casing.

The electric massage comb with the essential oil atomization and infrared light apparatus further includes a motor press cover for delimiting the sonic motor, where the motor press cover is connected to the bottom casing by using a screw.

A motor holder for accommodating the sonic motor is disposed on the bottom casing by one-piece molding, where a motor rubber cushion is disposed at an inner side of the motor holder, and a motor cover rubber cushion at an inner side of the motor press cover.

Preferably, the teeth plate is connected to the output shaft of the sonic motor by using a screw.

The electric massage comb with the essential oil atomization and infrared light apparatus further includes a protection cover at an outer side of the teeth plate, a teeth magnet is disposed on the teeth plate, a protection cover magnet is disposed on the protection cover, the protection cover and the teeth plate stick to each other through the teeth magnet and the protection cover magnet.

Preferably, a rechargeable battery assembly and a battery foam matching the rechargeable battery assembly are further disposed inside the main massage comb body.

The electric massage comb with the essential oil atomization and infrared light apparatus further includes an indicator light cover on the main massage comb body and an indicator light main board at an inner side of the indicator light cover.

Preferably, an air inlet joint and an air outlet joint are disposed at the air inlet and the air outlet parts of the cartridge body respectively, and straight-through hose joints are separately disposed at opening parts at two ends of the straight-through hose.

Preferably, the cartridge body assembly further includes support silica gel matching the cartridge support, heating strip silica gel, a heat-insulating foam, and lower cartridge casing silica gel gasket matching the lower cartridge casing.

A technical effect achieved in this application by resolving the technical problem is as follows:

Compared with the conventional technology, the present utility model provides the electric massage comb with the essential oil atomization and infrared light apparatus. The main massage comb body 25, the control circuit board 19 disposed inside the main massage comb body 25, the cartridge body assembly, the teeth plate 41 at the outer side of the main massage comb body 25, and the sonic motor 32 for driving the teeth plate 41 to vibrate are disposed together. The spray outlet for spraying mist is provided on the teeth plate part 41, and the cartridge body assembly, the sonic motor 32, and the control circuit board 19 are electrically connected. The electric massage comb with the essential oil atomization and infrared light apparatus further includes the air pump 21 that is disposed inside the main massage comb body 25 and that is interconnected to the cartridge body assembly through an air duct, where mist produced by the cartridge body assembly is transferred, through a straight-through hose 12 with pumping of the air pump 21, to the spray outlet part for spraying; and the teeth plate 41 is connected to an output shaft of the sonic motor 32 and is driven by the sonic motor 32 to vibrate. During actual application, the mist produced by the cartridge body assembly and air produced by the working air pump 21 are blown to hair and scalp through the air duct. As head moves during massage, atomized essential oil evenly covers scalp and hair to provide care for scalp and hair.

### BRIEF DESCRIPTION OF DRAWINGS

The FIGURE is a schematic diagram of an exploded view of a structure of an electric massage comb with essential oil atomization and infrared light apparatus according to the present utility model.

In the FIGURE: 1: cartridge body; 2: support silica gel; 3: ceramic heating strip support; 4: heating strip silica gel; 5: ceramic heating strip; 6: heat-insulating foam; 7: lower cartridge casing silica gel gasket; 8: lower cartridge casing; 9 and 14: magnet sheets; 10: conductive pin; 11: straight-through hose joint; 12: straight-through hose; 13: air inlet joint; 15: cartridge support; 16: air outlet joint; 18: top casing; 19: control circuit board; 21: air pump; 25: main massage comb body; 27: motor press cover; 28: indicator light cover; 29: indicator light main board; 30: motor cover rubber cushion; 32: sonic motor; 33: rechargeable battery assembly; 34: motor rubber cushion; 35:

battery foam; 36: infrared light board; 37: bottom casing; 38: spray joint; 40: teeth magnet; 41: teeth plate; 45: protection cover magnet; and 46: protection cover.

### DETAILED DESCRIPTION OF THE EMBODIMENTS

To make the purposes, technical solutions, and advantages of the present utility model clearer, the following further describes the present utility model in detail with reference to the accompanying drawings and embodiments. It should be understood that, specific embodiments described herein are merely used to explain the present utility model but not to limit the present utility model.

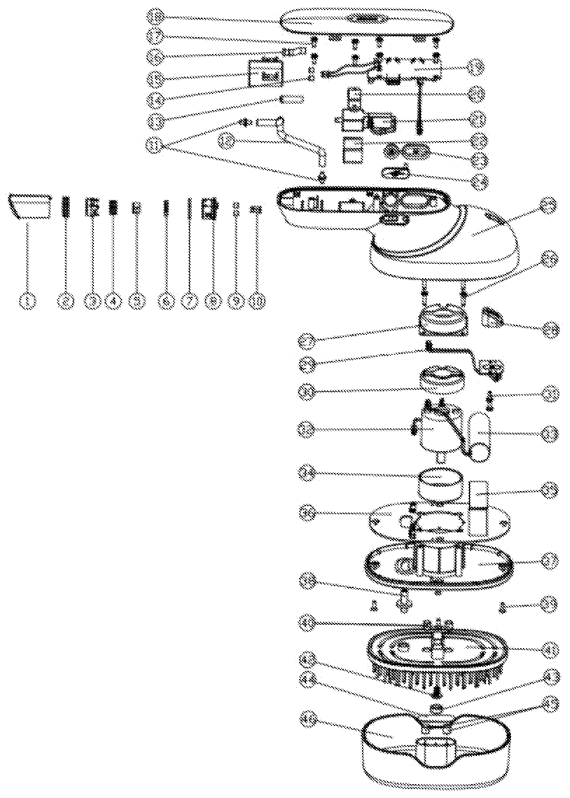

Refer to FIG. 1. The present utility model provides an electric massage comb 100 with an essential oil atomization and infrared light apparatus, including a main massage comb body 25, a control circuit board 19 disposed inside the main massage comb body 25, a cartridge body assembly, a teeth plate 41 at an outer side of the main massage comb body 25, and a sonic motor 32 for driving the teeth plate 41 to vibrate, where a spray outlet for spraying mist is provided on the teeth plate part 41, and the cartridge body assembly, the sonic motor 32, and the control circuit board 19 are electrically connected.

The electric massage comb with the essential oil atomization and infrared light apparatus further includes an air pump 21 that is disposed inside the main massage comb body 25 and that is interconnected to the cartridge body assembly through an air duct, where mist produced by the cartridge body assembly is transferred, through a straight-through hose 12 with pumping of the air pump 21, to the spray outlet part for spraying.

The teeth plate 41 is connected to an output shaft of the sonic motor 32 and is driven by the sonic motor 32 to vibrate.

In this application, the main massage comb body 25, the control circuit board 19 disposed inside the main massage comb body 25, the cartridge body assembly, the teeth plate 41 at the outer side of the main massage comb body 25, and the sonic motor 32 for driving the teeth plate 41 to vibrate are disposed together. The spray outlet for spraying mist is provided on the teeth plate part 41, and the cartridge body assembly, the sonic motor 32, and the control circuit board 19 are electrically connected. The electric massage comb with the essential oil atomization and infrared light apparatus further includes the air pump 21 that is disposed inside the main massage comb body 25 and that is interconnected to the cartridge body assembly through an air duct, where mist produced by the cartridge body assembly is transferred, through a straight-through hose 12 with pumping of the air pump 21, to the spray outlet part for spraying; and the teeth plate 41 is connected to an output shaft of the sonic motor 32 and is driven by the sonic motor 32 to vibrate. During actual application, the mist produced by the cartridge body assembly and air produced by the working air pump 21 arc blown to hair and scalp through the air duct. As head moves during massage, atomized essential oil evenly covers scalp and hair to provide care for scalp and hair.

The sonic motor 32 is connected to teeth to generate high-frequency vibration to massage head for an effect of reducing fatigue.

The sonic motor 32 gently transfers the vibration deep into skin, muscles, and joints to more effectively massage head for an effect of reducing fatigue and caring for scalp.

In some other embodiments, an infrared light board 36, or infrared light beads, is further disposed in the main massage comb body 25, to deepen the mist into scalp with infrared light radiation.

The infrared light board 36 is disposed at the bottom of the massage comb teeth. The light board is disposed with 20 beads of infrared light and 12 beads of far infrared light. With infrared and far infrared light radiation, head massage can deepen into scalp for improving microcirculation, enhancing body's metabolism, and boosting immunity.

With infrared and far infrared light radiation, head massage can deepen into scalp for improving microcirculation, enhancing body's metabolism, and boosting immunity.

The cartridge body assembly includes a cartridge body 1, a ceramic heating strip 5 inside the cartridge body 1, and a ceramic heating strip support 3 for fixedly disposing the ceramic heating strip 5, an air inlet and an air outlet are provided on the cartridge body 1 for a connection to the air pump 21 and mist pumping.

A microporous-ceramic atomization core is disposed inside the cartridge body assembly, and essential oil is canned in the cartridge body assembly for atomization through the microporous-ceramic atomization core. A conductive pin 10 is disposed at the bottom of the cartridge body assembly for a connection to a main board, and the main board controls the atomization.

A cartridge support 15 for mounting the cartridge body assembly is further disposed in the cartridge body assembly.

One end of the cartridge body 1 is disposed with a lower cartridge casing 8, and the cartridge body assembly further includes the conductive pin 10 on the lower cartridge casing part 8 and magnet sheets 9 and 14 for ensuring a conductive connection effect.

The magnets are disposed at both the bottom of the cartridge body assembly and a position of an essential oil cavity in the main body, to fix the cartridge body assembly to the main body and make an electric connection to the conductive pin 10.

A vertical groove corresponding to the spray outlet is provided on the teeth plate 41, and electric massage comb with an essential oil atomization and infrared light apparatus further includes a spray joint 38 disposed at the vertical groove part, where the spray joint 38 is interconnected to the cartridge body assembly through the straight-through hose 12.

The main massage comb body 25 includes a top casing 18 and a bottom casing 37, and the teeth plate 41 is disposed near the bottom casing 37.

The electric massage comb with the essential oil atomization and infrared light apparatus further includes a motor press cover 27 for delimiting the sonic motor 32, where the motor press cover 27 is connected to the bottom casing 37 by using a screw.

A motor holder for accommodating the sonic motor 32 is disposed on the bottom casing 37 by one-piece molding, where a motor rubber cushion 34 is disposed at an inner side of the motor holder, and a motor cover rubber cushion 30 at an inner side of the motor press cover 27.

The teeth plate 41 is connected to the output shaft of the sonic motor 32 by using a screw.

The electric massage comb with the essential oil atomization and infrared light apparatus further includes a protection cover 46 at an outer side of the teeth plate 41, a teeth magnet 40 is disposed on the teeth plate 41, a protection cover magnet 45 is disposed on the protection cover 46, the protection cover 46 and the teeth plate 41 stick to each other through the teeth magnet 40 and the protection cover magnet 45.

A rechargeable battery assembly 33 and a battery foam 35 matching the rechargeable battery assembly 33 are further disposed inside the main massage comb body 25.

The electric massage comb with the essential oil atomization and infrared light apparatus further includes an indicator light cover 28 on the main massage comb body 25 and an indicator light main board 29 at an inner side of the indicator light cover 28.

An air inlet joint 13 and an air outlet joint 16 are disposed at the air inlet and the air outlet parts of the cartridge body 1 respectively, and straight-through hose joints 11 are separately disposed at opening parts at two ends of the straight-through hose 12.

The cartridge body assembly further includes support silica gel 2 matching the cartridge support 15, heating strip silica gel 4, a heat-insulating foam 6, and lower cartridge casing silica gel gasket 7 matching the lower cartridge casing 8.

An operating principle of the vibration in this application is that: After a vibration function is enabled, the control circuit board 19 drives the sonic motor 32, and the sonic motor 32 drives the connected teeth to generate approximately high-speed small movements for head massage.

An operating principle of the essential oil atomization is that: After an atomization function is enabled, the control circuit drives the microporous-ceramic atomization core to heat the essential oil for atomization, and the control circuit board controls the air pump 21 to generate flowing air that enters the essential oil cartridge air inlet, to deliver the atomized essential oil to an opening at the bottom of the teeth through the air duct connected to the air outlet. As head moves during massage, the atomized essential oil evenly covers scalp and hair to provide care for scalp and hair.

An operating principle of the infrared light is that: After the product is turned on, the control circuit board 19 supplies power to the infrared light board, and the infrared light and the far infrared light on the light board are turned on for infrared light and far infrared light care for scalp during head massage.

A technical effect achieved in this application by resolving the technical problem is as follows:

Compared with the conventional technology, the present utility model provides the electric massage comb 100 with the essential oil atomization and infrared light apparatus. The main massage comb body 25, the control circuit board 19 disposed inside the main massage comb body 25, the cartridge body assembly, the teeth plate 41 at the outer side of the main massage comb body 25, and the sonic motor 32 for driving the teeth plate 41 to vibrate are disposed together. The spray outlet for spraying mist is provided on the teeth plate part 41, and the cartridge body assembly, the sonic motor 32, and the control circuit board 19 are electrically connected. The electric massage comb with the essential oil atomization and infrared light apparatus further includes the air pump 21 that is disposed inside the main massage comb body 25 and that is interconnected to the cartridge body assembly through an air duct, where mist produced by the cartridge body assembly is transferred, through a straight-through hose 12 with pumping of the air pump 21, to the spray outlet part for spraying; and the teeth plate 41 is connected to an output shaft of the sonic motor 32 and is driven by the sonic motor 32 to vibrate. During actual application, the mist produced by the cartridge body assembly and air produced by the working air pump 21 are blown to hair and scalp through the air duct. As head moves during massage, atomized essential oil evenly covers scalp and hair to provide care for scalp and hair.

The foregoing implementations of the present utility model do not constitute a limitation on the protection scope of the present utility model. Any modification, equivalent replacement, improvements, etc. made without departing from the spirit and principle of the present utility model shall all fall within the protection scope of the claims of the present utility model.

What is claimed is:

1. An electric massage comb with an essential oil atomization and infrared light apparatus, comprising a main massage comb body, a control circuit board disposed inside the main massage comb body, a cartridge body assembly, a teeth plate at an outer side of the main massage comb body, and a sonic motor for driving the teeth plate to vibrate, wherein a spray outlet for spraying mist is provided on the teeth plate part, and the cartridge body assembly, the sonic motor, and the control circuit board are electrically connected, wherein
the electric massage comb with the essential oil atomization and infrared light apparatus further comprises an air pump that is disposed inside the main massage comb body and that is interconnected to the cartridge body assembly through an air duct, wherein mist produced by the cartridge body assembly is transferred, through a straight-through hose with pumping of the air pump, to the spray outlet part for spraying; and
the teeth plate is connected to an output shaft of the sonic motor and is driven by the sonic motor to vibrate.

2. The electric massage comb with the essential oil atomization and infrared light apparatus according to claim 1, wherein an infrared light board, or infrared light beads, is further disposed in the main massage comb body, to deepen the mist into scalp with infrared light radiation.

3. The electric massage comb with the essential oil atomization and infrared light apparatus according to claim 1, wherein the cartridge body assembly comprises a cartridge body, a ceramic heating strip inside the cartridge body, and a ceramic heating strip support for fixedly disposing the ceramic heating strip, an air inlet and an air outlet are provided on the cartridge body for a connection to the air pump and mist pumping.

4. The electric massage comb with the essential oil atomization and infrared light apparatus according to claim 3, wherein a cartridge support for mounting the cartridge body assembly is further disposed in the cartridge body assembly; and
one end of the cartridge body is disposed with a lower cartridge casing, and the cartridge body assembly further comprises a conductive pin on the lower cartridge casing part and magnet sheets for ensuring a conductive connection effect.

5. The electric massage comb with the essential oil atomization and infrared light apparatus according to claim 1, wherein a vertical groove corresponding to the spray outlet is provided on the teeth plate, and electric massage comb with an essential oil atomization and infrared light apparatus further comprises a spray joint disposed at the vertical groove part, wherein the spray joint is interconnected to the cartridge body assembly through the straight-through hose.

6. The electric massage comb with the essential oil atomization and infrared light apparatus according to claim 1, wherein the main massage comb body comprises a top casing and a bottom casing, and the teeth plate is disposed near the bottom casing;
the electric massage comb with the essential oil atomization and infrared light apparatus further comprises a motor press cover for delimiting the sonic motor, wherein the motor press cover is connected to the bottom casing by using a screw; and
a motor holder for accommodating the sonic motor is disposed on the bottom casing by one-piece molding, wherein a motor rubber cushion is disposed at an inner side of the motor holder, and a motor cover rubber cushion at an inner side of the motor press cover.

7. The electric massage comb with the essential oil atomization and infrared light apparatus according to claim 1, wherein the teeth plate is connected to the output shaft of the sonic motor by using a screw; and
the electric massage comb with the essential oil atomization and infrared light apparatus further comprises a protection cover at an outer side of the teeth plate, a teeth magnet is disposed on the teeth plate, a protection cover magnet is disposed on the protection cover, the protection cover and the teeth plate stick to each other through the teeth magnet and the protection cover magnet.

8. The electric massage comb with the essential oil atomization and infrared light apparatus according to claim 1, wherein a rechargeable battery assembly and a battery foam matching the rechargeable battery assembly are further disposed inside the main massage comb body; and
the electric massage comb with the essential oil atomization and infrared light apparatus further comprises an indicator light cover on the main massage comb body and an indicator light main board at an inner side of the indicator light cover.

9. The electric massage comb with the essential oil atomization and infrared light apparatus according to claim 3, wherein an air inlet joint and an air outlet joint are disposed at the air inlet and the air outlet parts of the cartridge body respectively, and straight-through hose joints are separately disposed at opening parts at two ends of the straight-through hose.

10. The electric massage comb with the essential oil atomization and infrared light apparatus according to claim 4, wherein the cartridge body assembly further comprises support silica gel matching the cartridge support, heating strip silica gel, a heat-insulating foam, and lower cartridge casing silica gel gasket matching the lower cartridge casing.
